(19) United States
(12) Patent Application Publication
(10) Pub. No.: US 2012/0245486 A1
(43) Pub. Date: Sep. 27, 2012

(54) GHOST-CORE BIOPSY NEEDLE

(76) Inventors: Anthony Melchiorri, Normal, IL (US); Torsten Schreiber, Indianapolis, IN (US); Danielle N. Joaquin, Tinley Park, IL (US); Christopher Ferree, Bloomington, IN (US); Casey L. Brown, Bedford, IN (US); Allen E. Hacker, Bloomington, IN (US); James M. Carlson, Lafayette, IN (US)

(21) Appl. No.: 13/427,998

(22) Filed: Mar. 23, 2012

Related U.S. Application Data
(60) Provisional application No. 61/467,488, filed on Mar. 25, 2011.

(51) Int. Cl. A61B 10/02

(52) U.S. Cl. 600/567

(57) ABSTRACT

Among other things, there is disclosed embodiments of biopsy needles having improved radiological visualization properties. Exemplary needles include an outer cannula and a stylet slidable within the cannula. At least a portion of the stylet is radiologically contrasting to at least a part of the cannula. When the stylet is extended from the cannula within the patient, the clinician can observe radiologically where the extended portion of the stylet is, because of the radiological contrast between the stylet and cannula. The clinician can compare that contrasting area to the tissue of interest, to assure that the needle is properly placed.

### REFERENCE TO RELATED APPLICATION

This application claims the benefit of U.S. Provisional Patent Application No. 61/467,488, filed Mar. 25, 2011, which is hereby incorporated by reference.

The present disclosure concerns improvements in biopsy-needle technology that provides advantages in visualization and accurate sampling during a percutaneous biopsy procedure.

### BACKGROUND

It is known to acquire one or more samples of tissue when particular localized medical problems are suspected, in order to test such samples and determine whether or to what extent a problem exists. For example, if a physician discovers a growth within soft tissue of a patient and wishes to test whether it is benign or cancerous, a deep biopsy sample of the soft tissue can be acquired. For such cases, biopsy devices have been developed that can be passed through skin, muscle and/or other tissues or body-walls and into the area of tissue of concern to the physician. The device's distal end is used to cut out a sample of the suspicious tissue, which can then be withdrawn and analyzed.

There are a number of tools that have been used to obtain a sample of tissue from a patient, for testing and diagnosis of potential medical problems with the specific tissue or the patient in general. Attempts have been made to make the procedure as minimally-invasive as possible. To that end, needles have been developed which can be inserted through the skin of a patient directly or through the vasculature to a tissue mass or other particular location from which a sample is desired. Through mechanical or other means a sample is captured by the needle, and the needle is withdrawn with the sample.

Various forms of existing biopsy needles use a moving mandrel or cannula that facilitates movement through tissue and cutting and capturing a sample from surrounding tissue. A quick, longitudinal movement of a cannula over a mandrel or stylet, for example, is generally used to cut through tissue faster than the tissue can be moved forward or out of the way by the device. Depending on the particular type of device, problems that exist with such biopsy devices can include an incorrect placement or orientation of the device, resulting in obtaining a sample that is partially or completely from tissue not of interest to the physician. For instance, a stylet or cannula may be incorrectly located in the patient so that its cutting edge is in the middle of or beyond the tissue from which a sample is desired. When the stylet or cannula shoots forward to capture the sample, what is captured is little or none of the tissue of interest, but is rather other tissue outside that area.

To address that difficulty, technological solutions have been proposed. For example, it is known to use x-ray or fluoroscopy imaging to view the positioning of biopsy devices, which are generally made of the same metal throughout their insertion portions. Under x-ray imaging, for example, the metal of the needle can be observed compared to adjacent bone or other reference anatomical structure(s). Such a sampling end can be continuously observed as it moves through the patient, or discrete x-ray images may be taken, for example when the clinician believes the end of the needle is at the desired sampling location and/or at one or more intermediate locations. However, with needles and other biopsy devices having multiple pieces that interact to obtain the sample, each of those metal pieces are visible (and may overlap) under x-ray observation, making the visualization or reading of the image confusing or difficult. Moreover, such metal needles can produce artifacts or phantoms in the image, with multiple pieces each producing such effects, thus generating further confusion among parts, erroneous images and/or views that are difficult to sort out.

Another solution is to attempt to obtain a larger tissue sample, in the hope that more or all of the obtained tissue is from the area of interest. Larger biopsy needles or needles that obtain a full core of tissue (rather than smaller or more-limited samples) can address some inaccuracies in the physician's or other operator's placement. For example, if the placement is not well-centered with respect to the tissue of interest, a larger needle or full-core sampler may obtain more of that tissue than a smaller sampler or one that takes a smaller cross-sectional sample.

However, enlarging the needle's cross-section or the overall sample cross-section does not address difficulties in correctly placing the device, particularly longitudinally. For example, if the operator does not know with significant accuracy the depth of the tissue of interest, or does not assess correctly whether the tissue of interest is directly ahead of the cutting area of the device, the insertion of the device may result in its cutting area being well in front of or beyond the tissue of interest. A sample gathered in those conditions (an “undershoot” or “overshoot”) consequently includes tissue in front of or beyond the tissue of interest, resulting in acquisition of a significant proportion of tissue that does not provide the information the physician is seeking. Of course, it is also desirable to maintain a relative small size for such devices so as to minimize discomfort or additional damage to the patient's tissues when a biopsy sample is obtained.

Accordingly, there remains a need for a biopsy needle that is capable of obtaining a sample of a desired size while keeping the profile of the opening in the patient minimal, and the placement of which can be monitored or verified with greater ease and efficiency.

### SUMMARY

Among other things, there is disclosed a biopsy needle that includes an outer cannula and an inner stylet. In particular embodiments, the outer cannula has a distal end and is radiopaque at least at that distal end, and the stylet has a distal end and a notch adjacent its distal end. The entirety of the portion of the stylet that includes the notch and its distal end have a radiopacity different from that of the outer cannula's distal end, so that that portion of the stylet can be distinguished from the distal end of the cannula by virtue of the difference of the intensities of their respective images in a radiologic image when the stylet's distal end is extended beyond the distal end of the cannula.

The stylet may have an area between its distal end and the notch, and include a marker in that area. The marker has a radiopacity sufficient to be seen in a radiologic image, so that in a radiologic image a space can be seen between the marker and the cannula's distal end. In some embodiments, the marker is entirely inside of the stylet, and in others the marker can be a thin band around the outer circumference of the stylet. As one example, the marker may be in a plane substantially perpendicular to a longitudinal axis of the stylet. The portion of the stylet that includes the notch may be invisible under x-ray, computed tomography imaging, magnetic resonance imaging, or other types of imaging. At least the portion of the stylet including the notch can be a composite material having a strength-to-weight ratio equal to or greater than that of stainless steel.

Also disclosed is a needle for sampling tissue that includes an outer cannula having a distal end and being radiopaque, and an inner member having a distal end and notch adjacent the inner member's distal end. The notch extends longitudinally along the inner member a distance greater than the width of the inner member. The entirety of the portion of the inner member that contains the notch and the inner member's distal end is made of a composite material that is radiolucent and has a strength-to-weight ratio larger than stainless steel. The inner member includes a radiopaque marker attached between the notch and the inner member's distal end. The inner member is slidable within the outer cannula between a first position and a second position. In the first position, the inner member's notch is within the cannula's distal end, and in the second position the inner member's distal end and its notch extend from the cannula's distal end. When viewed under external imaging in that second position, an image shows contrast between the cannula and the inner member, which defines a space between the marker and the cannula's distal end in the image. The space defines the part of the inner member that includes the notch.

In some embodiments, the marker is a band extending around the circumference of the inner member in a plane substantially perpendicular to the inner member's longitudinal axis. The marker can also be enclosed by the inner member. Particular examples of the inner member are entirely made of the composite material, while others include a proximal portion of metal joined to the composite material distal end.

Methods regarding such devices are also disclosed. For example, a method of obtaining a sample of tissue of interest from a patient can include one or more of providing a sampling device having an outer cannula and an inner stylet member each having a respective distal end, with the inner member being movable through the outer cannula; inserting the device into the patient so that the distal ends of the outer cannula and inner member are within the patient; extending the distal end of the inner member into the patient beyond the distal end of the outer cannula; and after that extending step, viewing a radiologic image of the device within the patient and observing a gap in the image between the distal end of the inner member and the distal end of the outer cannula.

Such methods can also include determining whether the gap is within the tissue of interest for sampling, and if it is not within the tissue of interest for sampling, repositioning the device. The repositioning can include additional occurrence(s) of the viewing and/or observing steps. In other embodiments, the viewing step includes observing whether the tissue of interest is visible in the gap. If so, the device is fired to move the cannula forward over the inner member to capture tissue between the cannula and inner member. If not, the device can be repositioned and/or the viewing step repeated.

Thus, it will be understood that embodiments of biopsy needles and methods for their use are disclosed. In general, an inner stylet having a notch that can extend from an outer cannula is provided. The stylet is at least partially of a material of different radiological viewing characteristics as compared to the material of the cannula, so that a contrast between the stylet and cannula can be radiologically observed when the stylet and notch are extended outside of the cannula. If a radiopaque marker is provided at or adjacent to the stylet's distal end, then the marker and cannula border a gap or other radiologically-contrasted area, which defines where the notch is. Such embodiments work to limit or remove errors in placement of cutting tips of such needles, and thus reduce the need to reposition or take second or further samples in order to obtain a sufficient measure of the tissue of interest.

### BRIEF DESCRIPTION OF THE DRAWINGS

FIG. 1 is a top plan view of an embodiment of a biopsy needle according to this disclosure.

FIG. 2 is a side elevational view of an inner member used in the embodiment of FIG. 1.

FIG. 3A is a top plan view of part of the embodiment of FIG. 1 in an extended or primed condition.

FIG. 3B is a side elevational view of the part shown in FIG. 3A.

FIG. 4A is a top plan view of the embodiment of FIG. 1 in a pre-cocked or unstressed condition.

FIG. 4B is a top plan view of the embodiment of FIG. 1 in a cocked condition.

FIG. 4C is a top plan view of the embodiment of FIG. 1 in an extended or primed condition.

FIG. 5 is a representation of a radiologic image of a portion of the embodiment of FIG. 1 within a patient and in an extended or primed condition.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated device, and further applications of the principles of the disclosure as illustrated therein are herein contemplated as would normally occur to one skilled in the art to which the disclosure relates.

Referring now generally to the drawings, there is shown an embodiment of a biopsy needle 20. Needle 20 includes an inner stylet 22, an outer cannula 24 and a handle 26. Stylet 22 is slidable within cannula 24, and both are connected to and operable by handle 26 in this embodiment, as will be further explained below.

Stylet 22 is substantially cylindrical in the illustrated embodiment, having an elongated body 32 extending between a proximal portion 34 (which connects to handle 26) and a distal end 36. Elongated body 32 is at least substantially circular in the illustrated embodiment for ease of use and manufacture. Distal end 36 is sharpened in the illustrated embodiment so as to provide a forward surface that assists in moving tissue out of the way during insertion of needle 20 into the patient. For example, a surface 38 that is planar and oblique to the longitudinal axis of cannula 22 can be cut, ground or otherwise formed in distal end 36.

Proximal of surface 38 there is formed in stylet 22 a notch 40. In the illustrated embodiment, notch 40 has a depth to a substantially flat inner surface 42 of approximately half of the diameter of stylet 22 or less, and end surfaces 44 perpendicular to or forming an obtuse angle with surface 42. It will be understood that other embodiments of notch 40 can be of greater or lesser depths may be hollowed out, and/or can have end surface(s) 44 oriented with an acute angle between surfaces 44 and 42. Notch 40 is provided so that tissue to be biopsied can enter it and be cut off and contained in notch 40, as further discussed below. Between surface 44 at the distal end of notch 40 and surface 38 there is in this embodiment a short area 46 of stylet 22 that has the same configuration as stylet 22 proximal of notch 40. Area 46, in an embodiment as shown that has a substantially circular cross section for stylet 22, has a similarly circular circumference that is continuous.

Stylet 22 has at least a portion that is radiolucent in this embodiment so as to improve the visibility of tissue and the determination of the precise location of the sampling area of needle 20 when it is within the patient. The illustrated embodiment of stylet 22 has a substantially uniform radiolucent material throughout, extending from distal end 36 through all of proximal portion 34. It is a different material from that used for cannula 24, which is of a rigid metal (e.g. stainless steel) in particular embodiments for better cutting of tissue. A contrast under x-ray, CT or other imaging is therefore presented between stylet 22 and cannula 24.

A particular embodiment of stylet 22 is formed from a single piece of radiolucent material, which is initially-formed to include end surface 38 and notch 40 as indicated above. For example, beginning with a one-piece extruded cylinder of radiolucent material, portions of the material may be cut or otherwise removed from the piece to form end surface 38 and notch 40. In other embodiments, at least the portion of stylet 22 that includes end surface 38, all of notch 40, and any further portion of stylet 22 that extends from cannula 24 during use is made of such radiolucent material (e.g. the portion shown extending from cannula 24 in FIGS. 3A, 3B). That radiolucent portion P may be fixed to a proximal portion 34 of stylet 22 that is of a metal, synthetic or other rigid material, making a hybrid stylet 22. In either case, all of the portion of stylet 22 that extends from cannula 24 during use of needle 20 is of a radiolucent material in this embodiment.

It has been found that synthetic composite materials that are biocompatible, invisible to x-ray and computed tomography (CT) imaging, and have a strength-to-weight ratio equal to or higher than that of stainless steel are well-suited to a radiolucent stylet 22 that resolves imaging problems inherent in existing biopsy needles. A particular example of such a synthetic composite material is used in tubing sold under the trademark POLYMED® (Polygon Company, Walkerton, Ind.), which has been found to be biocompatible and invisible under x-ray and CT imaging, yet has strength and other physical properties that allow manufacture into and operation as stylet 22 without significant loss of performance over stainless steel, and also has high torsion stability, that is, it maintains shape under twisting or torsion. In the biopsy-needle context, twisting of the needle on insertion and use is common, and therefore many thin synthetic materials without significant torsion stability may not be suitable. Such materials provide the strength needed to combat buckling as stylet 22 is moved through tissue (including bony or cartilaginous tissue), as discussed below. It is recognized that existing biopsy needles using two parts are generally made of a single metal material. Use of metals is indicated for their rigidity in pressing through bodily tissue. Use of one metal for multiple parts provides for ease in manufacturing and also avoids the possibility of different electrical potentials between two different metals in the body, which can affect or be affected by bodily processes.

Radiolucent materials that do not have the strength-to-weight ratio indicated above might be used for some or all of stylet 22, and would provide many of the advantages noted. However, if a substance is substantially weaker than metals, then in order to provide the physical strength needed for stylet 22 to operate as desired, a thicker or wider-diameter stylet may be provided from such materials. Expanding the size of the stylet, and therefore of the entire needle insertion portion, is less desirable, not least because of the added discomfort to the patient. Further, a stylet having a larger outer diameter (for structural stiffness) yet with the same area for the notch, and thus a smaller notch portion per unit outer diameter, has an increased uncertainty of positioning and obtains a smaller sample per unit diameter. That is, guaranteeing where the relatively-smaller notch is in relation to the tissue of interest is more difficult for a larger-outer-diameter device.

These problems are not realized in the case of the disclosed radiolucent stylet 22, of electromagnetically transparent material as noted above for example. An identically- or similarly-sized stylet 22 of such a material operates just as well or better than a stainless steel stylet, and has been discovered to provide substantial benefits in use with a metal cannula, and can be made to fit existing examples of cannulas in existing needle types without a reduction in stiffness or other structural properties needed for holding or containing tissue during a biopsy procedure. In the present disclosure, no increase in size or thickness of stylet 22 is required with the beneficial addition of imaging contrast between stylet 22 and cannula 24, as would be expected with less sturdy materials.

The illustrated embodiment of stylet 22 is entirely of a radiolucent material as described above, which can be extruded to the size and shape of stylet 22, and when cured can be beveled and cored to form notch 40 and its adjacent edges 44. Accordingly, manufacture of stylet 22 is generally much easier and requires less energy than forming and cutting metal stylets. Further, errors in manufacturing stylet 22 can be easily dealt with by recycling the extrudable material, rather than by scrapping faulty or irregular metal items. It will be seen, however, that advantages of the present disclosure are also realized in embodiments that are partially of a radiolucent material as described above or similar material. For example, the portion beginning at distal end 36 of stylet 22 and ending beyond notch 20 of stylet 22 may be of the radiolucent sturdy material, and that portion may be attached to a stainless steel or other metal medial portion that is connected to handle 26. While such embodiments may be somewhat more difficult to manufacture, the visualization benefits discussed herein will still be available.

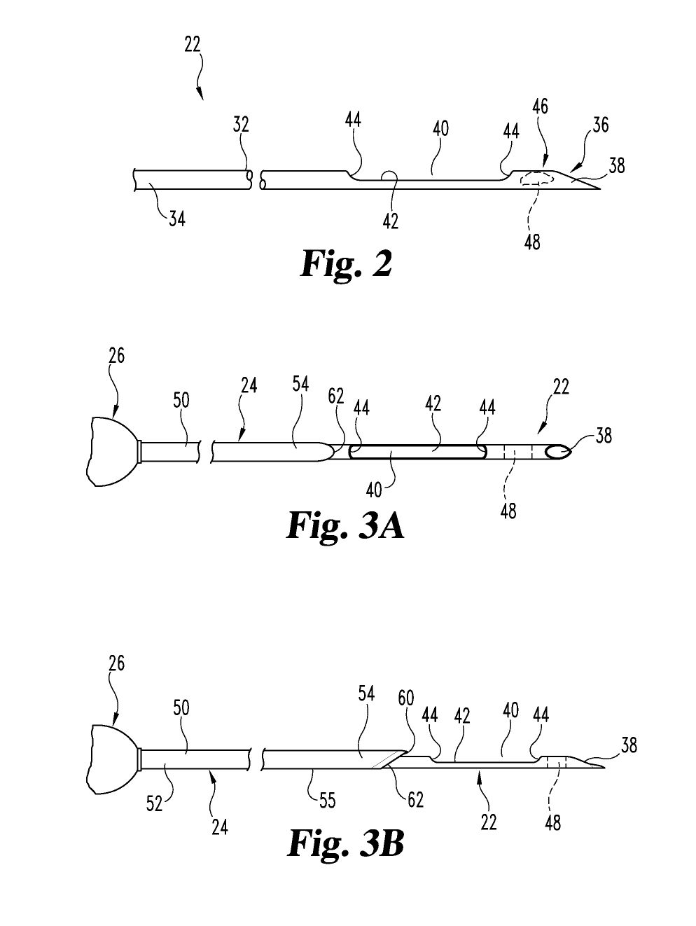

In addition, the illustrated embodiment of stylet 22 includes a radiopaque marker 48 in or around area 46 of stylet 22. In FIG. 2, marker 48 is a roughly oblate or spherical body embedded within stylet 22 in area 46. The entirety of marker 48 is between end surface 38 and surface 44 of notch 40, so that there is no interference by marker 48 with the tissue-cutting or -moving role of surface 38 or with the filling of notch 40 with tissue. Further, by positioning marker 48 as noted, marker 48 defines at least approximately both the distal end of notch 40 and the distal end surface 38 of stylet 22. In FIGS. 3A and 3B, marker 48 is a thin band around the exterior circumference of area 46. The band is generally in a plane perpendicular to the longitudinal axis of stylet 22, and again is between and non-interfering with end surface 38 and notch 40. Examples of materials for markers 48 are noble metals such as gold or silver. As will be discussed further below, markers 48 are specifically placed and/or oriented to show very clearly under x-ray or CT imaging a point at or beyond where notch 40 ends, as well as the point or an approximation of the point where distal end 38 of stylet 22 is.

The illustrated embodiment of cannula 24 is tubular, having an elongated body 50 extending between a proximal portion 52 and a distal end 54. Elongated body 50 is of substantially the same cross-sectional shape as stylet 22 in some embodiments. Body 50 is at least substantially circular in the illustrated embodiment, having a cylindrical outer surface 55 and an inner cylindrical surface defining a lumen, for ease of use and manufacture. Proximal end 52 is connected to handle 26, as will be further discussed below. Tubular body 50 is cut obliquely at distal end 54 to form a surface 60 with one or more sharp edges 62. In the illustrated embodiment, an essentially planar oblique section is taken through cannula 24, so that end surface 60 and edge(s) 62 are formed. In the illustrated embodiment, the cut distal end 54 is beveled or otherwise shaped to an edge 62 so as to cut tissue as cannula 24 is advanced. In the illustrated embodiment, surface 60 is formed so that the relatively uppermost portion of cannula 24 (as seen in FIGS. 3A-3B and as generally inserted into the patient) is distal-most.

As previously indicated, cannula 24 is of a radiopaque material, such as stainless steel or other biocompatible metal. In addition to providing the sturdiness and sharp distal end 54 needed to effectively obtain a tissue sample, cannula 24 (and particularly the portion extending proximally from distal end 54) provides a bright image under x-ray, CT or other imaging, and a substantial contrast to the lack of image provided under such imaging by the notch portion of stylet 22. It will also be understood that cannula 24 can be made of non-metallic materials, so that needle 20 can be used in conjunction with magnetic resonance imaging (MRI). A suitable example of such a material is a composite having a strength-to-weight ratio equal to or greater than that of stainless steel, as indicated above. Such a material not only forestalls buckling during use, as noted above, but also provides the strength needed for the good cutting edge(s) 62 of cannula 24. Proper radiopaque markers may also be included in embodiments of cannula 24 that are non-metallic.

Handle 26 is connected to each of stylet 22 and cannula 24 at their respective proximal portions, so that stylet 22 is within cannula 24, and so that stylet 22 and cannula 24 are slidable with respect to each other. Examples of structures usable as part of handle 26 and cannula 24 are those currently used with QUICK-CORE® products sold by Cook Medical (Bloomington, Ind.). Embodiments of structure suitable for use herein are shown in U.S. Provisional Application No. 61/261,857, filed on Nov. 17, 2009, and in U.S. Provisional Application No. 61/412,625, filed on Nov. 11, 2010, the entirety of both of which are incorporated herein by reference.

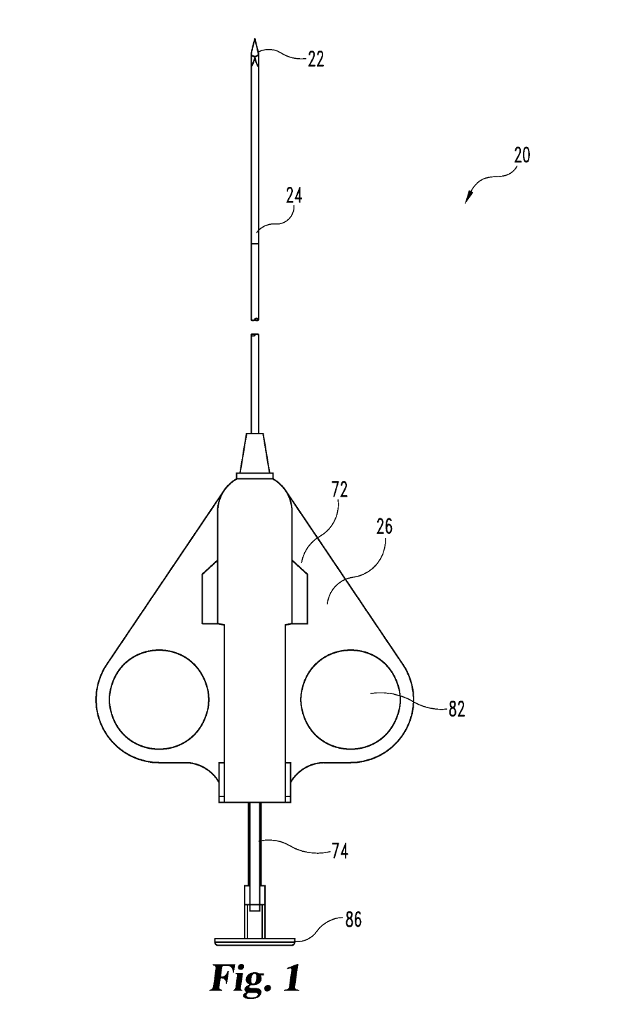

Handle 26, in the embodiment of FIG. 1, includes a housing 72 and a trigger or actuator 74. Housing 72 includes finger holds 82 which are substantially circular in this embodiment, and within which actuator 74 is slidable forward and backward (proximally and distally). When assembled, handle 26 is cocked by pulling actuator 74 (e.g. via grip 86) out or away from housing 72. As one example, pulling grip 86 proximally moves both cannula 24 and stylet 22 together, maintaining their respective tips at approximately the same location, to maintain a cocked position.

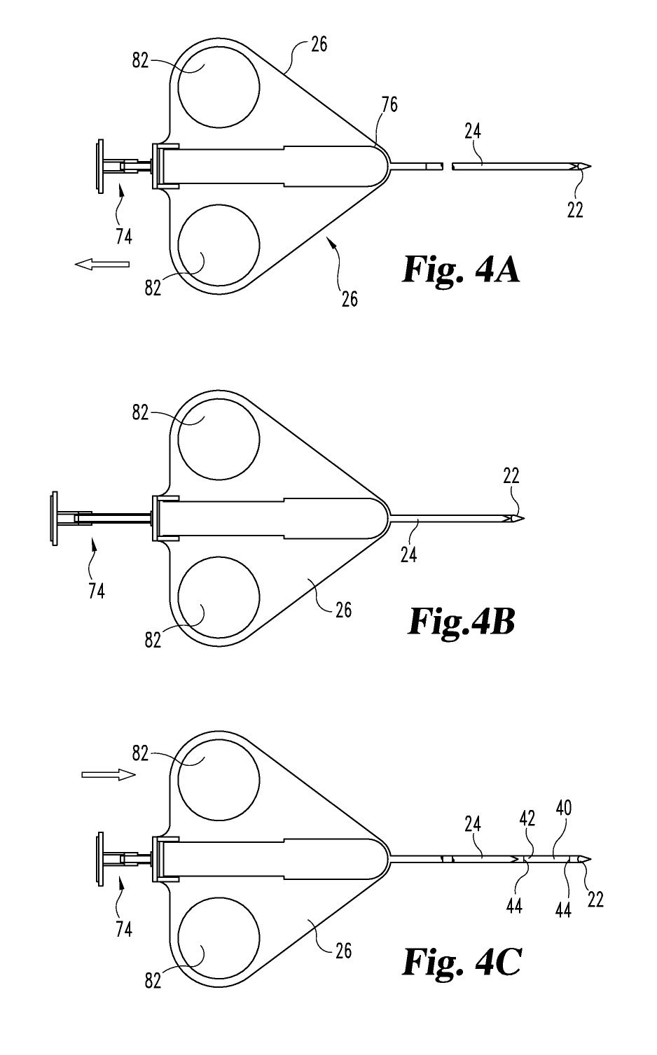

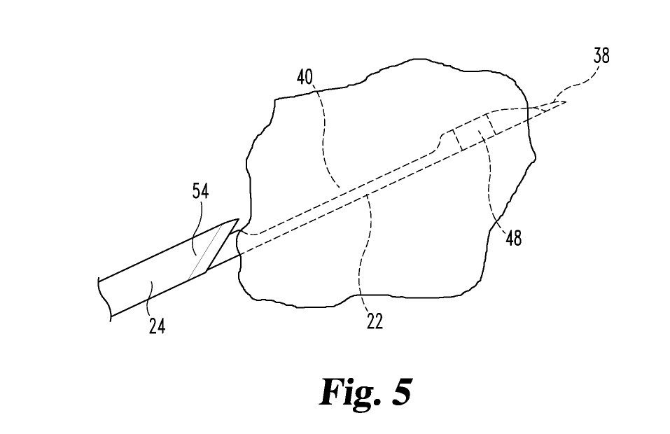

Once needle 20 is cocked, the user pushes grip 86 to move actuator 74 (and thus stylet 22) forward a distance sufficient to prime needle 20 by moving notch 40 of stylet 22 out of the distal end of cannula 24 (e.g. FIGS. 3A, 3B, 4C). Such forward movement of actuator 74 moves stylet 22, but does not move cannula 24. When notch 40 is fully exposed from cannula 24, e.g. when proximal surface 44 adjacent notch 40 is just beyond end 54 of cannula 24, further forward movement of actuator 74 (and stylet 22) is impeded by handle 26. As will be explained further below, this priming step is performed once needle 20 has been inserted into the patient so that distal ends of stylet 22 and cannula 24 are in or almost in the tissue to be sampled. The priming step operates to expose notch 40 from cannula 24 and to allow tissue into notch 40.

Extending stylet 22 from cannula 24 so as to expose notch 40 provides a clear contrast or differentiation between stylet 22 and cannula 24 when visualized, e.g. under x-ray or CT imaging. As stylet 22 is extended from cannula 24, only marker 48 at the distal end of stylet 22 can be seen under such imaging. The portion of stylet 22 between marker 48 and the distal end 54 of cannula 24 cannot be seen through such imaging. Accordingly, the priming step creates a space in an image between marker 48 and end 54 of cannula 24, which demarcates where notch 40 of stylet 22 is. The tissue can also be seen in that space, so that it is evident to the observer that notch 40 is juxtaposed to the tissue of interest, especially if the tissue has been previously marked with a radio-contrast agent or other indicator. The clear contrast under imaging between stylet 22 (or the portion of it that includes notch 40) and cannula 24 is a substantial advantage over existing devices.

Needle 20 is fired to capture tissue within notch 40. Pushing grip 86 further toward housing 72 overcomes the impedance and allows a loaded spring to thrust cannula 40 forward relative to stylet 22. Cannula 24 travels quickly through tissue and over notch 40 of stylet 22, capturing tissue within notch 40, between cannula 24 and stylet 22. Thus, firing handle 26 propels cannula 24 over stylet 22 to sever and trap tissue within notch 40 of stylet 22. The illustrated embodiment of needle 20 is a single action biopsy device which is effective when used to obtain tissue samples.

Device 20 thus permits a cocking step that prepares for firing cannula 24 over and along stylet 22, an insertion step in which the relatively positioned stylet 22 and cannula 24 are inserted into the body, and a firing step in which cannula 24 is released to move forward rapidly over stylet 22 and return to or toward the pre-cocking or unstressed state. The cocking step is performed by holding finger holds 82 and pulling back on actuator 74 until it clicks as discussed above. Inserting needle 20 is accomplished while holding and applying force to handle 26, forcing stylet 22 and cannula 24 forward into the body. Pushing actuator 74 forward gently following insertion moves notch 40 out from cannula 24, and allows tissue into notch 40. Firing cannula 24 is accomplished by pushing forward actuator 74 to overcome the cocked state, and may be accomplished by the same hand that holds finger grips 82 in this embodiment. The firing propels cannula 24 over stylet 22 and through tissue, trapping a length of tissue in notch 40. The steps noted above may be applied in a different order, as may be indicated by the clinical situation.

Cannula 24 and stylet 22 are slidable with respect to each other, as indicated above. Stylet 22 extends through the lumen of outer cannula 24. In a particular embodiment, the outer diameter of inner cannula 22 is approximately the same as the inner diameter of outer cannula 24, so that there is little play or space between cannulas 22 and 24, yet they can move smoothly with respect to each other. Stylet 22 and cannula 24 have a first relative position (e.g. FIG. 4A) which is seen before cocking or after firing needle 20. In that first relative position, stylet 22 and cannula 24 are approximately coextensive in the illustrated embodiment, i.e. distal ends of stylet 22 and cannula 24 are at approximately the same location.

The use of needle 20 will now be described in the context of obtaining a sample of soft tissue for testing purposes. It will be understood that methods for obtaining samples of other tissues or for other purposes are also contemplated.

The surgeon, clinician or other medical professional first determines a location in a patient, with its depth under the skin, from which a tissue sample is desired. In one embodiment, stylet 22 and cannula 24 of needle 20 are initially in the relative position indicated in the example of FIG. 4A. The user then cocks needle 20, as noted above. In the cocked state, in this embodiment, stylet 22 and cannula 24 are in a relative position exemplified in FIG. 4B, with ends of stylet 22 and cannula 24 substantially coinciding and retracted from the position of FIG. 4A.

In that state, the user places distal ends 36 and/or 54 of stylet 22 and/or cannula 24 against the skin at a place proximate to the desired location, and inserts needle 20. Needle 20 forces a path through the skin and subcutaneous tissue to a point in or just before the location from which a sample is to be taken. The path size and shape is determined by the outer configuration of cannula 24. In embodiments in which cannula 24 is very thin, the path is not substantially larger than the outer diameter of stylet 22, reducing discomfort from the biopsy procedure. Stylet 22 prevents or impedes tissue entry into cannula 24 during the insertion, since stylet 22 has an outer diameter that is substantially the same as the inner diameter of cannula 24. The strength-to-weight ratio of the radiolucent material of stylet 22 is relatively high, as noted above, so that pressure of tissue on end 36 of stylet 22 during insertion does not buckle or otherwise damage stylet 22.

The insertion may be made under continuous CT, fluoroscopy, or other observation, assuming the administration of such continuous observation provides small risk or is otherwise warranted. Alternatively, the insertion may be made with periodic single images taken to monitor the progress of the needle, or with at least one image taken when the needle is believed to be in the desired location. Cannula 24 is metal and therefore is plainly shown in the image(s). The clinician consults the image(s) to determine whether distal end 54 of cannula 24 is adjacent to or within the area of tissue to be sampled. Relative location of distal end 54 of cannula 24 may be made easier by applying a radiopaque substance (e.g. a barium salt) to the tissue of interest (e.g. a lump). If the image(s) indicate that distal end 54 of cannula 24 is not in a desired location, needle 20 may be adjusted, as by inserting further toward or into the tissue of interest. Additional image(s) may be taken to verify that the adjusted needle is in the desired location. At this point in this embodiment, with stylet 22 essentially entirely within cannula 24, only cannula 24 can be seen in such image(s).

With needle 20 in the desired location and in the cocked state (e.g. with distal ends 54, 36 of cannula 24 and stylet 22 even with each other), needle 20 is primed as indicated above. That priming step advances stylet 22 out of cannula 24 so that notch 40 is beyond distal end 54 of cannula 24 (e.g. FIGS. 3A, 3B, 4C). Notch 40 generally faces tissue to be sampled, and the recoil of such tissue results in tissue entering notch 40. Radiolucent stylet 22 does not show on x-ray, CT or similar images, except for marker 48. During or after the priming of needle 20, image(s) of needle 20 may be taken. A representation of such an image is shown in FIG. 5. The observer of the image will see marker 48 and cannula 24, with a space showing tissue in between marker 48 and distal end 54 of cannula 24. The presence of stylet 22 and its invisibility or other contrast with respect to cannula 24 under imaging is indicated by the dotted lines in FIG. 5. As indicated above, stylet 22 is constructed in the illustrated embodiment so that notch 40 is essentially bounded by marker 48 distally and by end 54 of cannula 24 proximally (i.e. little or nothing of stylet 22 proximal of notch 40 extends from cannula 24 in the primed state). The observer thus knows from the space between marker 48 and cannula 24 precisely where notch 40 is, and can compare that to the surrounding tissue to ensure that notch 40 is within the tissue of interest. No imaging artifacts are present from stylet 22, reducing confusion or difficulty in reading the image(s). The radiolucent quality of stylet 22 provides an absence or reduction of image, and of reflections or other artifacts from it that could appear in an image, between marker 48 and cannula 24 that defines where notch 40 is located.

When stylet 22 is fully-advanced, with tissue in notch 40, the user may verify the placement as indicated above. When the user is satisfied that the notch is placed in the tissue of interest, the user fires cannula 24 forward through the throw-length. As cannula 24 moves forward, its forward edge 62 cuts through tissue, trapping it in notch 40 of stylet 22. Cannula 24 returns to the initial position with its end 54 substantially at the same position as end 36 of stylet 22.

After firing, with stylet 22 and cannula 24 back in the extended relative position indicated in FIG. 4A, needle 20 is withdrawn. Once needle 20 is withdrawn, the tissue sample is removed by cocking and priming needle 20, as indicated above, to expose notch 40 and the tissue within it. The tissue can be extracted using a forceps or other tool, or in some embodiments by inverting notch 40 and allowing the tissue to drop out of notch 40 into a specimen dish or other container. If the user determines that additional sample(s) are needed, then the procedure above can be repeated to obtain such samples.

In the illustrated embodiment, stylet 22 has a close fit with cannula 24 within its lumen 58, and the two are slidable with respect to each other. By having both a “close fit” and slidability, it is meant that there is no substantial separation or gap between stylet 22 and cannula 24, as by a boss or flange. As seen in the embodiments in the drawings, stylet 22 and cannula 24 have a close and slidable fit at least along their respective distal ends, and in some embodiments that close and slidable fit extends along all or substantially all of one or both of their respective lengths. Such a configuration minimizes the external size of a needle needed to obtain a particular amount of tissue.

Needle 20 makes it much easier for the clinician to verify that the sampling area (i.e. notch 40) is in the proper location relative to the tissue to be sampled without weakening the needle's sampling area. That ease reduces the likelihood of erroneous misplacement of a biopsy needle, which can result in a sample being taken of undesired tissue, and the need of a second or further samples. In some systems, a new sample requires a new needle, so that averting such errors can cut costs in terms of using additional product as well as in terms of additional time for the clinician and time and discomfort for the patient.

As previously noted, current biopsy needles use all-metal construction, of a single material, to ensure visibility under CT or x-ray imaging. The inserted component(s) have that visibility advantage on initial insertion, but do not and cannot show under such imaging the precise location of the sampling area. In fact, the inventors have noted that such needles create false images or artifacts on a CT or x-ray image that can indicate that the needle (or part of it) is located in tissue in a position other than its actual position, thus preventing accurate placement and sampling. Only when the sample is retrieved can it be discovered that the tissue of interest was not sampled or was incompletely sampled.

The examples discussed above note the radiolucency of stylet 22 or at least a portion of it. In particular embodiments, radiolucency (i.e. where the visualized portion of stylet 22 does not appear on an x-ray, CT or similar image) is used. Such a stylet 22 may be said to provide the largest or most substantial contrast under imaging with respect to cannula 24, since cannula 24 shows up with intensity and stylet 22 is invisible. Of course, as indicated above the contrast under imaging between cannula 24 and stylet 22 is advantageous. It will be understood that materials that have such a contrast compared to the metal of cannula 24 and the strength traits needed for a biopsy needle can be used, even if the material is viewable to some extent under the relevant imaging. Accordingly, as used herein “radiolucent” refers not only to materials invisible to imaging systems such as x-ray and/or CT scanning, but also to those having a significant and observable contrast under such imaging compared to metal.

While the disclosure has been illustrated and described in detail in the drawings and foregoing description, the same is to be considered as illustrative and not restrictive in character, it being understood that only embodiments have been shown and described and that all changes, equivalents, and modifications that come within the spirit of the disclosures defined by the following claims are desired to be protected. Particular features described with respect to one embodiment or structure are usable with other embodiments or structures disclosed herein.

What is claimed is:

1. A biopsy needle, comprising:
an outer cannula having a distal end, said outer cannula being radiopaque at least at said distal end;
an inner stylet having a distal end and a notch adjacent said stylet distal end, the entirety of the portion of said stylet that includes said notch and said stylet distal end having a radiopacity different from that of said outer cannula distal end, so that said portion of said inner stylet can be distinguished from said distal end of said cannula by virtue of the difference of the intensities of their respective images in a radiologic image when said distal end of said stylet is extended beyond said distal end of said outer cannula.

2. The biopsy needle of claim 1, wherein said stylet has an area between its distal end and said notch, and wherein said needle includes a marker in said area, said marker having a radiopacity sufficient to be seen in a radiologic image, so that in a radiologic image a space can be seen between said marker and said distal end of said outer cannula.

3. The biopsy needle of claim 2, wherein said marker is entirely inside of said stylet.

4. The biopsy needle of claim 2, wherein said marker is a thin band around the outer circumference of said stylet, said marker being in a plane substantially perpendicular to a longitudinal axis of said stylet.

5. The biopsy needle of claim 1, wherein said portion of said stylet including said notch is invisible under x-ray or computed tomography imaging.

6. The biopsy needle of claim 5, wherein at least said portion of said stylet including said notch is a composite material having a strength-to-weight ratio equal to or greater than that of stainless steel.

7. The biopsy needle of claim 6, wherein at least a portion of said cannula is of said composite material, wherein said needle is usable with magnetic resonance imaging.

8. A needle for sampling tissue, comprising:
an outer cannula having a distal end, said outer cannula being radiopaque;
an inner member having a distal end and notch adjacent said inner member distal end, said notch extending longitudinally along said inner member a distance greater than the width of said inner member, the entirety of the portion of said inner member that includes said notch and said inner member distal end being made of a composite material that is radiolucent and has a strength-to-weight ratio larger than stainless steel, said inner member having a radiopaque marker attached between said notch and said inner member distal end, said inner member being slidable within said outer cannula between a first position and a second position,
wherein in said first position said notch is within said cannula distal end, and in said second position said inner member distal end and said notch extend from said cannula distal end, and
wherein when viewed under external imaging in said second position, an image shows contrast between said cannula and said inner member, defining a space between said marker and said cannula distal end in the image, said space defining the part of said inner member including said notch.

9. The needle of claim 8, wherein said marker is a band extending around the circumference of said inner member in a plane substantially perpendicular to the longitudinal axis of said inner member.

10. The needle of claim 8, wherein said marker is enclosed by said inner member.

11. The needle of claim 8, wherein said inner member is entirely of said composite material.

12. The needle of claim 8, wherein said inner member includes a proximal portion of metal joined to said composite material distal end.

13. A method of obtaining a sample of tissue of interest from a patient, comprising:
providing a sampling device having an outer cannula and an inner stylet member each having a respective distal end, said inner member movable through said outer cannula;
inserting said device into the patient so that said distal ends of said outer cannula and inner member are within the patient;
extending said distal end of said inner member into the patient beyond said distal end of said outer cannula;
after said extending step, viewing a radiologic image of said device within said patient and observing a gap in the image between said distal end of said inner member and said distal end of said outer cannula.

14. The method of claim 13, further comprising determining whether said gap is within the tissue of interest for sampling, and if said gap is not within the tissue of interest for sampling, repositioning said device.

15. The method of claim 14, wherein said repositioning includes an additional occurrence of said viewing and observing steps.

16. The method of claim 13, wherein said viewing step includes observing whether the tissue of interest is visible in said gap.

17. The method of claim 16, further comprising firing said device to move said cannula forward over said inner member to capture tissue between said cannula and said inner member if the tissue of interest is visible in said gap.

18. The method of claim 16, further comprising repositioning said device if the tissue of interest is not visible in said gap and repeating said viewing step.
